(19) United States
(12) Patent Application Publication
(10) Pub. No.: US 2026/0199119 A1
(43) Pub. Date: Jul. 16, 2026

(54) Therapeutic Body Cover

(71) Applicant: Blyth Meechan, Cranbourne, Victoria (AU)

(72) Inventor: Blyth Meechan, Cranbourne, Victoria (AU)

(21) Appl. No.: 19/109,531

(22) PCT Filed: Sep. 1, 2023
(86) PCT No.: PCT/AU2023/050853

§ 371 Date: Mar. 6, 2025

(30) Foreign Application Priority Data
Sep. 9, 2022 (AU) 2022902608

(51) Int. Cl. A61F 5/37

(52) U.S. Cl. CPC A61F 5/3723

(57) ABSTRACT

A therapeutic body cover (n00) made of stretchable and resilient material. The cover comprises (i) a body tube (n10); (ii) a top end (n20) including a neck (n22) and a top opening (n24) for a wearer's head or face and having an upper inner bearing surface (n26) to retain the top end at or around the wearer's head or shoulders; and (iii) a lower end (n50) comprising a closable lower opening (n54) including a lower inner bearing surface (n56) to retain the lower. The length of the unstretched tube from the upper inner bearing surface to the lower inner bearing surface is adapted to position the lower end at or above the wearer's ankles to reduce the risk of the lower end being a tripping hazard in an upright or standing position. The lower opening is at a lowermost edge (n58) of the tube.

### CROSS REFERENCE TO RELATED APPLICATIONS

This application is a national phase entry under 35 U.S.C. § 371 of International Patent Application PCT/AU2023/050853, filed Sep. 1, 2023, designating the United States of America and published in English as International Patent Publication WO 2024/050595 on Mar. 14, 2024, which claims the benefit under Article 8 of the Patent Cooperation Treaty to Australian Patent Application Serial No. 2022902608, filed Sep. 9, 2022, the entireties of which are hereby incorporated by reference.

### FIELD OF INVENTION

This invention relates to a therapeutic body cover. More particularly, this invention relates to a therapeutic body cover adapted to selectively cover a wearer's limbs. Still more particularly, this invention relates to a therapeutic body cover adapted to selectively cover a wearer's limbs and compress the wearer's body.

### BACKGROUND ART

The following references to and descriptions of prior proposals or products are not intended to be, and are not to be construed as, statements or admissions of common general knowledge in the art. In particular, the following prior art discussion should not be assumed to relate to what is commonly or well known by the person skilled in the art, but to assist in the inventive process undertaken by the inventor(s) and in the understanding of the invention.

AU2021245252 (Mundt) describes a body compression sleepwear with a leg opening comprised of a pouch formed in a foot end and including an opening located at around the upper ankle position. Because of the stretchable nature of the fabric, negotiating the opening to exit one's feet can be awkward and the wearer's feet tend to get trapped in the pouch. Moreover, a sit to stand action can be hazardous as the wearer's feet may be restricted, making the wearer prone to tripping.

An object of the present invention is to ameliorate one or more of the aforementioned disadvantages of the prior art or to at least provide a useful alternative thereto. This invention may be aimed at providing a therapeutic body cover for providing comfort and security to a wearer prone to injury who is momentarily unable to control the movements of their limbs.

### STATEMENT OF INVENTION

The invention according to one or more aspects is described herebelow and further defined in the independent claims. Some optional and/or preferred features of the invention are described herebelow and further defined in the dependent claims.

Accordingly, in one aspect of the invention there is provided:

A thereapeutic body cover made of stretchable and resilient material and adapted to:
selectively cover a wearer's limbs; and
lightly compress the wearer's body,
the cover comprising:
a tube optionally of variable unstretched breadth along its length from top to bottom to cover the full length of the wearer;
a top end including an opening for a wearer's head or face and having an upper inner bearing surface to retain the top end at or around the wearer's head or shoulders; and
a lower end comprising an opening adapted to be closable to cover the wearer's lower limbs and to present a lower inner bearing surface to retain the lower end at or around the lowermost extent of the wearer's lower limbs;
wherein:
the releasable opening is adapted to be opened by the wearer to allow one or both of the wearer's lowermost limbs to exit the opening; and
the length of the unstretched tube from the upper inner bearing surface to the lower inner bearing surface, and the elasticity of the material, is adapted to lift the lower end above the wearer's ankles to reduce the risk of the lower end being a tripping hazard in an upright position; and
the opening is at the lowermost edge of the tube.

### Body Cover

The body cover is therapeutic in that its purpose is directed at harm-mitigation for persons who experience epileptic and other seizures that can make a person vulnerable to physical trauma from uncontrolled movement of limbs. Persons with an epileptic condition may experience seizures in which their limbs can rapidly move to the endangerment of both the person and those around them. The therapeutic body cover addresses both the need for a compressive calming effect and the option for the wearer to extend one's lower limbs from the lower opening either for cooling relief or to facilitate safe walking or standing in an upright position.

Other therapeutic uses include for comfort therapy for persons on the autism spectrum and emotional reassurance for those who have suffered emotional trauma, whereby a compression garment can provide a calming effect. The body cover also be useful for general mental health as it can be improve sleep patterns in otherwise healthy individuals by providing a sense of security and comfort.

The tube extends from the top end to the lower end and may be formed from a sheet of material joined along opposed longitudinal extending side edges to form a flexible tube with a longitudinal seam. The tube may be, in front plan, a truncated fusiform or barrel shape. The unstretched upper and lower diameters or widths of the neck and the lower edge may be smaller than the maximum unstretched diameter or width of an intermediate cross-section of the tube. The upper diameter or width may be larger than that of the lower diameter or width, respectively. Alternatively, the lower diameter or width may be larger than that of the upper diameter or width, respectively.

The material preferably includes cotton and/or bamboo in a blend. The material may include polyester in the blend. The material may include Lycra® in the blend. The material may include stretch Sherpa or polar fleece, or other materials suitable for a cold-climate, in the blend. Preferably, the blend includes cotton and/or bamboo. The choice of material used depends on prevalent temperature and humidity conditions in the climate in which the body cover is used. Both cotton and bamboo can be used in hotter climates because of their light weight and breathability properties. Cotton fleecy forms may be used in colder climates where insulation against heat loss is desirable. The blend may be woven to contribute to the strength, tear/penetration-resistance and elasticity of the material.

In terms of environmental aspects, the impact of making the body cover out of particular materials also impacts the environmental footprint of manufacture per unit made. Whereas bamboo does not require as much consumption of water up to harvesting, bamboo requires more water than cotton to process into usable garment material during the material manufacturing phase. On balance, a higher content of bamboo is preferred for hot climate uses, whereas in relative terms, a higher content of cotton is desirable in colder climate applications.

### Top End

The top end may include an open neck or a hood combined with a neck. The neck may be smaller in diameter or width than the adjacent and connected section of tube. The transition between the neck and the adjacent section of tube may include a shoulder either side of the neck. The shoulder may extend laterally to meet an arm hole or sleeve. The wearer may have the option of extending one's upper limbs out of the arm holes or sleeves, or may, for safety and therapy, retain their upper limbs within the tube, not extending them out of the arm hole or sleeve. Alternatively, the wearer may have the option of extending their upper limbs out of the arm holes or sleeves for cooling or utility (to use one's hands).

### Lower End

The lower most edge may define the closable lower opening. The lower opening may be adapted to be releasably sealed. The releasable closure may be adapted to allow the wearer to break the seal. The breakage of the seal may be reversible by simply re-engaging the releasable closure to re-seal the closable lower opening. The releasable closure may be adapted to allow the wearer to break the seal using the wearer's lower limbs. The wearer is preferably able to push through the releasable closure to change the releasable closure from a closed state to an open state. The wearer is preferably able to push through to disengage one or more fasteners.

The lower inner bearing surface may be provided by the lower ban. More positively, the lower inner bearing surface may be provided when the releasable closure is sealed (against exiting of the wearer's lower limbs). The seal may be in the form of a closed structure provided by the releasable closure. For those with an autism condition, such compression of the wearer's body may provide a therapeutic effect. Moreover, for a wearer with an epileptic condition, the body cover provides gentle containment, mild restraint or other assistance in providing safe protection of the wearer's limbs during an epileptic episode.

The fasteners may include any one or a combination of two or more of the following non-exhaustive list of fasteners:
(1) Hook and loop strips. Each strip may extend along approximately half of the inner circumference of the lower band, so that the respective hook and loop strips oppose one another to adhere thereto and seal the lower opening. However, although hook and loop strips are functionally useful to seal the lower opening, because of the unpleasant tactile sensation on a wearer's lower limbs, hook and loop strips are not a preferred form of the fasteners;
(2) Press studs. The fasteners may include a series of one or more, preferably 2-4, most preferably 3, pairings of male and female press studs. Advantageously, the front side of the lower band includes one of male or female studs, and the rear side of the lower band includes the other of male or female studs, so that the members of the stud pairings are opposed and engageable to each other. The wearer may easily disengage one or more stud pairings to partially or fully open the lower opening to allow the wearer to extend one or more of their lower limbs out of the lower opening;
(3) Zip. A zip may extend along part or all of the lower end to retain the wearer's lower limb(s) when the releasable closure is in the closed state. The zip may include a zip slider with a pull tag. The pull tag may be operable by the wearer's toe or other part of their lower limb to undo the zip.
(4) Magnets. The fasteners may be in the form of magnetic strips or tabs. The magnetic strips or tabs may be useful to allow the wearer to disengage opposed magnetic strip or tabs pairings on the inner face of the lower band using their lower limbs to push through to open the lower opening. The magnetic fasteners are advantageously self-closing whereby opposed pairings are (a) attracted to each other, and (b) once in close proximity with their opposed member pair, will tend to engage each other and seal or partially seal the lower opening.

In a preferred form, the releasable closure includes 2-4, preferably 3, pairs of tab fasteners, that may be either press studs, magnets or other fastener tab pairings. Each pair of fasteners may be positioned and opposed so that, whilst opposed members of different pairs may be less advantageously attached, the fastener members of the first fastener pairing have a tendency, or are best positioned, to engage with each other. Similarly, the second, third or fourth pairs of fasteners are each respectively positioned and opposed so that their member fastener parts have a tendency to engage with the other corresponding member fastener part.

In terms of positioning, in the tab form of the fasteners, each of the tabs are spaced from each other along the length of the front side or the rear side. The spacing between each adjacent tab is preferably equal, but the spacing between adjacent member fastener parts may vary, as long as the spacing of the opposed corresponding member fastener parts is mirrored.

The bamboo component of the blend of material facilitates breathing of the material. Moreover, the lower opening enables a wearer to extend one or both of their lower limbs through the lower opening to provide a cooling option, for example, to allow heat dissipation and lowering of their body core temperature.

The wearer may use the body cover as sleepwear. The wearer may also choose to stand, whereby, if not for the releasable closure, the lower end would present a trip hazard to the wearer. Therefore, the ability for the wearer to easily disengage the releasable closure to allow their feet, legs or lower limbs to extend therethrough provides a hazard prevention feature.

### BRIEF DESCRIPTION OF THE DRAWINGS

The invention may be better understood from the following non-limiting description of preferred embodiments, in which;

FIG. 2) 1 is an upper perspective view of an upper portion of a therapeutic body cover according to a first embodiment;

FIG. 4) 2 is a schematic side view of the upper portion of the therapeutic body cover shown in FIG. 1;

FIG. 5) 3 is a lower perspective view of a lower portion of a therapeutic body cover according to a second embodiment;

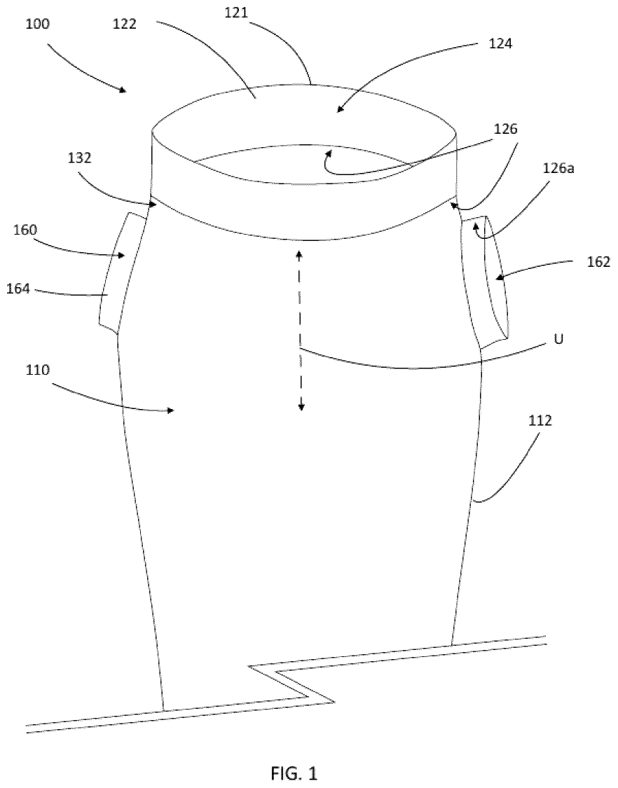

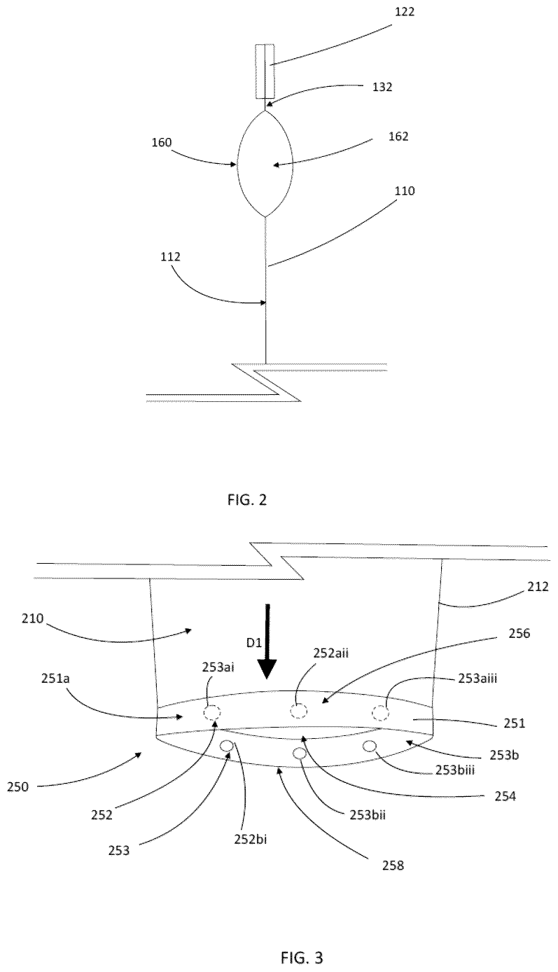

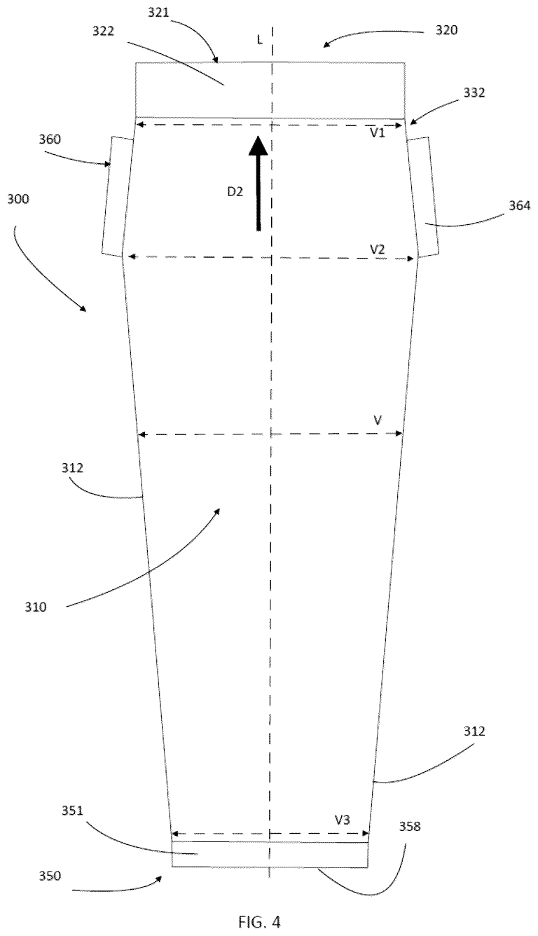

FIG. 1) 4 is a front view of a therapeutic body cover according to a third embodiment of invention; and

FIG. 3) 5 is a perspective view of the therapeutic body cover shown in FIG. 1.

### DETAILED DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention. In describing the various embodiments of the invention, like features will be referred to using like references, with references for features of each embodiment generally preceded by 1, 2, 3, or followed by a Roman numeric sequence, such as i, ii, iii, etc. or an alphabetical sequence such as a, b, c, relative to the corresponding feature of the first embodiment. For example, a feature 10 of the first embodiment may represented as 110, 210, 310, (or n10), or 10a, 10b, 10c, (or 10x) or 10i, 10ii, 10iii, (or 10r) etc. in second, third and fourth embodiments, respectively.

A therapeutic body cover (n00) is shown in the drawings and is made of stretchable and resilient material. The cover includes: (i) a body tube (n10); (ii) a top end (n20) including a neck (n22) and a top opening (n24) for a wearer's head or face and having an upper inner bearing surface (n26) to retain the top end at or around the wearer's head or shoulders; and (iii) a lower end (n50) comprising a closable lower opening (n54) adapted to be closable by a releasable closure (n52) to cover the wearer's lower limbs and to present a lower inner bearing surface (n56) to retain the lower end at or around the lowermost extent of the wearer's lower limbs. The lower inner bearing surface is opposed to the top inner bearing surface so that a lower force applied axially and outwardly to the lower inner bearing surface is in an opposite direction (D1) to an upper force (D2) applied axially and outwardly to the upper inner bearing surface. The therapeutic body cover is adapted to (a) selectively cover a wearer's limbs at the discretion of the wearer; and (b) lightly compress the wearer's body, generally conforming to the wearer's body shape, permitting limited movement, but restricting expansive limb movement. The lower opening is adapted to be opened by the wearer to allow part or all of one or both of the wearer's lowermost limbs to exit the lower opening. The length of the unstretched tube from the upper inner bearing surface to the lower inner bearing surface is adapted to position the lower end at or above the wearer's ankles to reduce the risk of the lower end being a tripping hazard in an upright or standing position. The lower opening is at a lowermost edge (n58) of the tube.

The therapeutic body cover (n00) is generally in the form of a tube including the tube body (n10). The body cover (n00) has a variable unstretched breadth (V) along its length from top to bottom to cover the full length of the wearer. The body tube (n10) is barrel shaped, frusto-elliptical, truncated-conical, having a larger central diameter than the diameter of the top and lower end portions (n20,n50). The body cover (n00) is bisymmetrical along a central longitudinal axis (L).

The body tube (n10) may be formed by joining a pair of substantially identical (in plan) front and rear sheets that are joined along spaced side seams (n12).

The greatest unstretched breadth is the mid-unstretched breadth (V2) of the body cover (n00) which is at chest level (C) and is closer to the top end (n20) than the lower end (n50) to accommodate the wider girth of a typical wearer around the shoulders and arms. The variable unstretched breadth (V) of the body cover (n10) tapers from the chest level (C) toward the lower end (n58) where the lower unstretched breadth (V3) is at its smallest. The variable unstretched breadth (V) tapers toward the top end (n20). The upper unstretched breadth (V1) is adjacent the neck (n22) and is greater than the lower unstretched breadth (V3), but less than the mid-unstretched breadth (V2).

The unstretched breadth or resting form of the body cover (n00) corresponds to the body cover (n00) when it is not stretched, for example, when it is not being worn, is laid out, folded or stored, etc.

The stretchable material of which the body cover (n00) is comprised includes a blend of any one or more of bamboo, polyester and Lycra®.

The body tube (n10) extends from the neck (n22) to the lower end (n50). The lower end (n50) is advantageously hemmed in a double layer to form a lower band (n51) that has greatest resilience and resistance to stretching compared to the portion of the body cover (n00) corresponding to the body tube (n10). The lower band (n51) is in the form of a skirt that terminates at the lower end (n50) in the lowermost edge (n58).

The lower most edge (n58) defines the closable lower opening (n54). The lower opening (n54) is adapted to be releasably sealed. The releasable closure (n52) is adapted to allow the wearer to break the seal, which breakage is reversible by simply re-engaging the releasable closure (n52) to re-seal the closable lower opening (n54). The releasable closure (n52) is adapted to allow the wearer to break the seal using the wearer's lower limbs to push through the releasable closure (n52) to disengage one or more fasteners (n53).

The fasteners (n53) may include any one or a combination of two or more of the following non-exhaustive list of fasteners (n53):
(1) hook and loop strips, each strip extending along approximately half of the inner circumference of the lower band (n51), so that the respective hook and loop strips oppose one another to adhere thereto and seal the lower opening (n54). However, although hook and loop strips are functionally useful to seal the lower opening (n54), because of the unpleasant tactile sensation on a wearer's lower limbs, hook and loop strips are not a preferred form of the fasteners (n53);
(2) Press studs. The fasteners (n53) may include a series of one or more, preferably 2-4, most preferably 3, pairings of male and female press studs. Advantageously, the front side (n251a) of the lower band (n51) includes one of male or female studs, and the rear side (n251b) of the lower band (n51) includes the other of male or female studs, so that the members of the stud pairings are opposed and engageable to each other. The wearer may easily disengage one or more stud pairings to partially or fully open the lower opening (n54) to allow the wearer to extend one or more of their lower limbs out of the lower opening (n54);
(3) Zip. A zip may extend along part or all of the lower end to retain the wearer's lower limb(s) when the releasable closure is in the closed state. The zip may include a zip slider with a pull tag. The pull tag may be operable by the wearer's toe or other part of their lower limb to undo the zip.
(4) Magnets. The fasteners (n53) may be in the form of magnetic strips or tabs. The magnetic strips or tabs may be useful to allow the wearer to disengage opposed magnetic strip or tabs pairings on the inner face of the lower band (n51) using their lower limbs to push through to open the lower opening (n54). The magnetic fasteners are advantageously self-closing whereby opposed pairings are (a) attracted to each other, and (b) once in close proximity with their opposed member pair, will tend to engage each other and seal or partially seal the lower opening (n54).

In FIG. 3, there are shown 3 pairs of tab fasteners (253ai-252biii), that may be either press studs, magnets or other fastener tab pairings. In the preferred form, the fasteners (n53) are press studs. The pair of fasteners (253ai,bi) are positioned and opposed so that, whilst opposed members of different pairs (designated with suffix ii or iii) may be less advantageously attached, the fastener members of the first fastener pairing (253ai,253bi) have a tendency to engage with each other. Similarly, the pair of fasteners (253aii,bii) are positioned and opposed so that they have a tendency to engage with each other. In like manner, the pair of fasteners (253aiii,biii) are positioned and opposed so that they have a tendency to engage with each other.

In terms of positioning, in the tab form of the fasteners (n53), each of the tabs are spaced from each other along the length of the front side (n251a) or the rear side (n51b). The spacing between each adjacent tab is preferably equal.

The bamboo component of the blend of material facilitates breathing of the material. Moreover, the lower opening (n54) enables a wearer to extend one or both of their lower limbs through the lower opening (n54) to provide a cooling option, for example, to allow heat dissipation and lowering of their body core temperature.

The wearer may use the body cover (n00) as sleepwear. The wearer may also choose to stand, whereby, of not for the releasable lower opening (n54), the lower end (n50) would present a trip hazard. Therefore, the ability for the wearer to easily disengage the releasable closure (n52) to allow their legs to extend therethrough provides a hazard prevention feature.

The top end (n20) may optionally include a hood attached to an upper edge (n21) of the neck (n22) or the upper edge (n21) may be a free end and have no attachment. Alternatively the hood may be detachable and attachable using a similar fastener system to that described with reference to the releasable closure (n52).

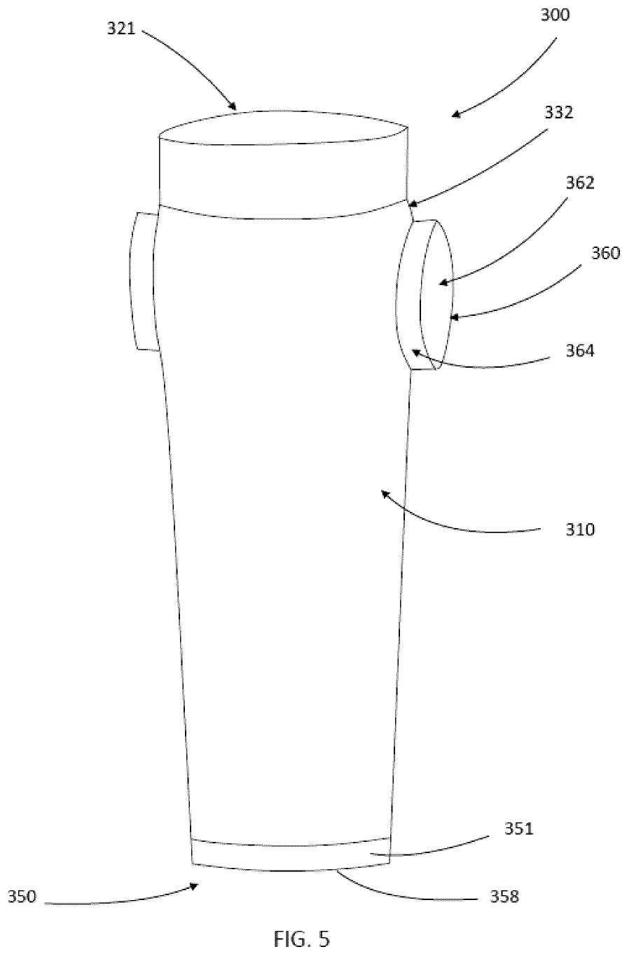

The neck (n52) forms an upper band. The neck (n52 is preferably formed by a double layer of material. The same material may be used for all components of the body cover (n00) with the exception of the releasable closure (n52). Adjacent the lower edge of the neck (n22) at the upper breadth (V1), the greater stretchability of the single layer of body tube (n10) material compared to the double layer of material of the neck (n22) may accommodate the shoulders of a wearer. That is, the single layer of material at the shoulder (n32) stretches more easily to allow space within the body cover (n00) for the wearer's shoulders. In any case, as shown in FIG. 5, the shoulder (332) may be shaped in the unstretched form of the body cover (n00) to shape the outline of the body cover (n00) to accommodate the wearer's shoulders, in combination with the body cover's (n00) capacity to stretch to fit the wearer's body.

In a preferred form of the invention, the body cover (n00) includes a pair of arm holes or sleeves (n62) immediately below the shoulders (n32) that are defined by a sleeve band (n64) that is formed from a double layer of material in the same manner as for the lower band (n51) or the neck (n22). Although permanently open, the arm holes (n62) are relatively small and together with remainder of an upper portion (U) of the body tube (n10), the upper portion (U) provides gentle containment, mild restraint or otherwise assists in providing safe protection of the wearer's upper limbs, when the wearer's upper limbs are contained within the body cover (n00). However, the wearer may choose to extend one or both of their upper limbs through the arm holes (n62) for comfort, cooling or to free their hands or upper limbs for utility.

The shoulders (n32) provide at least part of the upper inner bearing surface (n26). Advantageously, when the wearer's arms are extended through the arm holes (n62), the upper inner surface (n26a) of the sleeves (n62) forms a positive bearing surface that further resists the pulling of the sleeves (n62) and the neck (n22) downwardly. The wearer's shoulders will bear against the upper inner bearing surface (n26) when one or more of the wearer's lower limbs are retained and bear against the lower inner bearing surface (n56).

The lower inner bearing surface (n56) is provided by the lower band (n51) and, more positively when the releasable closure (n52) is sealed (against exiting of the wearer's lower limbs), by the closed structure provided by the releasable closure (n52). For those with an autism condition, such compression of the wearer's body may provide a therapeutic effect. Moreover, for a wearer with an epileptic condition, the body cover (n00) provides gentle containment, mild restraint or other assistance in providing safe protection of the wearer's limbs during an epileptic episode.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

In the present specification, terms such as “apparatus”, “means”, “device” and “member” may refer to singular or plural items and are terms intended to refer to a set of properties, functions or characteristics performed by one or more items or components having one or more parts. It is envisaged that where an “apparatus”, “means”, “device” or “member” or similar term is described as being a unitary object, then a functionally equivalent object having multiple components is considered to fall within the scope of the term, and similarly, where an “apparatus”, “assembly”, “means”, “device” or “member” is described as having multiple components, a functionally equivalent but unitary object is also considered to fall within the scope of the term, unless the contrary is expressly stated or the context requires otherwise. In the present specification, the phrase “and/or” refers to severally or any combination of the features. For example, the phrase “feature 1, feature 2 and/or feature 3” includes within its scope any one of the following combinations: Feature 1 or feature 2 or feature 3; feature 1 and feature 2 or feature 3; feature 1 or feature 2 and feature 3; feature 1 and feature 3 or feature 2; feature 1 and feature 2 and feature 3.

The meaning of descriptive, precise or absolute terms such as “flexed”, “normal”, “parallel”, “horizontal”, “vertical” or “fully” includes the preceding qualifier “substantially or almost”, unless the context or contrary is expressly indicated.

Qualifying relative terms, such as “relatively”, “sufficiently”, “near”, “almost” or “substantially”, may be taken to indicate a variation in an absolute value of between 0° and 10° or between 0% and 10%, relative to the absolute value. For example, “near horizontal” may mean any orientation between 0° and 10° relative to the horizontal.

Where the word “for” is used to qualify a use or application of an object term, the word “for” is only limiting in the sense that the device or component should be “suitable for” that use or application.

In the present specification, the term “integral” means formed of one body in a single process. In particular, the term “integrally formed” means formed of the one body without post-forming attachment of separately formed component parts. That is, “integrally formed” and the similar term “unitarily formed” mean formed in a single forming process and do not include post-forming attachment of component parts by means of fastener or other component fixing substances or methods.

Orientational terms used in the specification and claims such as vertical, horizontal, top, bottom, upper and lower are to be interpreted as relational and are based on the premise that the component, item, article, apparatus, device or instrument will usually be considered in a particular orientation, with the upper end uppermost and the lower end lowermost. Therefore, upper and upward are associated with the upper end and lower and downward are associated with the lower end.

The term “uppermost” is associated with the top end in the sense that it is a relative term, wherein “uppermost” or “upper” and like terms refer to the head end of a prostrate or prone wearer oriented substantially horizontally in a lying down pose, and “lowermost” or “lower” and like terms refer to the lower limb end of the wearer.

The term “lower limb” will generally refer to a wearer's foot, unless the wearer is an amputee, etc. and “arms” may refer to a one or both of a wearer's upper limbs.

In the present specification, the term “integral” means formed of one body in a single process. In particular, the term “integrally formed” means formed of the one body without post-forming attachment of separately formed component parts. That is, “integrally formed” and the similar term “unitarily formed” mean formed in a single forming process and do not include post-forming attachment of component parts by means of fastener or other component fixing substances or methods.

The articles “a” and “an” are used herein to refer to one or to more than one (i.e. to at least one) of the grammatical object of the article. By way of example, “an element” means one element or more than one element.

It will be appreciated by those skilled in the art that many modifications and variations may be made to the methods of the invention described herein without departing from the spirit and scope of the invention. The features and components of each of the embodiments of the invention described in the detailed description and/or depicted in the accompanying drawings may be interchangeable as required, with regard to functional equivalency and compatibility. A feature or component described with reference to one but not all embodiments, if functionally and dimensionally compatible as an addition with another embodiment herein described, or substitutable with a corresponding feature or component of that other embodiment in relation to which it has not been expressly described, should be read as a potential addition or substitution to that other embodiment and as being within the scope of the invention. Furthermore, in considering a feature or component that is described in relation a particular embodiment but may be omitted from the embodiment without losing the functionality characterising the invention and without departing from the scope of the invention, unless the context and expressions used in describing the embodiment imputes that the feature or component is essential to the invention as broadly described, the omittable feature or component may be read as not being included in the embodiment.

1. A therapeutic body cover made of stretchable and resilient material,
the cover comprising:
a body tube;
a top end including a neck and a top opening for a wearer's head or face and having an upper inner bearing surface to retain the top end at or around the wearer's head or shoulders; and
a lower end comprising a closable lower opening adapted to be closable by a releasable closure to cover the wearer's lower limbs and to present a lower inner bearing surface to retain the lower end at or around the lowermost extent of the wearer's lower limbs,
wherein:
the lower inner bearing surface is opposed to the top inner bearing surface so that a lower force applied axially and outwardly to the lower inner bearing surface is in an opposite direction to an upper force applied axially and outwardly to the upper inner bearing surface;
the therapeutic body cover is adapted to:
(a) selectively cover the wearer's lower limbs at the discretion of the wearer;
(b) selectively cover the wearer's upper limbs at the discretion of the wearer; and
(c) lightly compress the wearer's body, generally conforming to the wearer's body shape, permitting limited movement, but restricting expansive limb movement;
the lower opening is adapted to be opened by the wearer to allow part or all of one or both of the wearer's lowermost limbs to extend from the lower opening;
the length of the unstretched tube from the upper inner bearing surface to the lower inner bearing surface is adapted to position the lower end at or above the wearer's ankles to reduce the risk of the lower end being a tripping hazard in an upright or standing position;
the lower end is hemmed to form a lower band defining the lower opening; and
the lower band is in the form of a skirt that terminates at the lower end at a lowermost edge, the lower opening being at the lowermost edge of the tube.

2. The therapeutic body cover according to claim 1, wherein the cover is generally in the form of a tube including the tube body.

3. The therapeutic body cover according to claim 1, wherein the tube body has a variable unstretched breadth along its length from top to bottom to cover the full length of the wearer, the body tube is barrel-, frusto-elliptical-, or truncated-conical-shaped, having a larger central diameter than the diameter of the top and lower end portions and the body cover is bisymmetrical along a central longitudinal axis.

4. The therapeutic body cover according to claim 1, wherein the body tube is formed by joining a pair of substantially identical, in plan, front and rear sheets that are joined along spaced side seams.

5. The therapeutic body cover according to any one claim 1, wherein the body tube is formed from a single sheet that is folded over, so that the fold line forms one side of the body tube and the two free side edges of the single sheet are joined along part or all of their length to form a side seam.

6. The therapeutic body cover according to claim 3, wherein the greatest unstretched breadth of the body tube is the mid-unstretched breadth of the body cover which is at chest level and closer to the top end than the lower end to accommodate the wider girth of a typical wearer around the shoulders and arms.

7. The therapeutic body cover according to claim 3, wherein the variable unstretched breadth of the body cover tapers from the chest level toward the lower end where the lower unstretched breadth is at its smallest and the variable unstretched breadth tapers toward the top end, the upper unstretched breadth being adjacent the neck and greater than the lower unstretched breadth, but less than the mid-unstretched breadth.

8. The therapeutic body cover according to claim 1, wherein the stretchable material of which the body cover is comprised includes a blend of any two or more of bamboo, polyester and elastane.

9. The therapeutic body cover according to claim 1, wherein the body tube extends from the neck to the lower end.

10. The therapeutic body cover according to claim 1, wherein the lower end is hemmed in a double layer to form a lower band that has greatest resilience and resistance to stretching compared to the portion of the body cover corresponding to the body tube.

11. The therapeutic body cover according to claim 1, wherein the lower most edge defines the closable lower opening.

12. The therapeutic body cover according to claim 1, wherein the lower opening is adapted to be releasably sealed.

13. The therapeutic body cover according to claim 1, wherein the releasable closure is adapted to allow the wearer to open the releasable closure by breaking a reversible seal, which breakage is reversible by re-engaging the releasable closure to re-seal the closable lower opening.

14. The therapeutic body cover according to claim 13, wherein the releasable closure is adapted to allow the wearer to break the seal using the wearer's lower limbs to push through the releasable closure to disengage one or more fasteners.

15. The therapeutic body cover according to claim 14, wherein the fasteners include press studs comprising two or more pairs of tab fasteners.

16. The therapeutic body cover according to claim 1, wherein the top end includes a hood releasably attached to an upper edge of the neck.

17. The therapeutic body cover according to claim 1, wherein the body cover includes a pair of arm holes immediately below a pair of shoulders of the body cover.

18. The therapeutic body cover according to claim 17, wherein the arm holes are defined by a sleeve band that is formed from a double layer of material

19. The therapeutic body cover according to claim 17, wherein the shoulders provide at least part of the upper inner bearing surface that resists the pulling of the neck downwardly, the wearer's shoulders adapted to bear against the upper inner bearing surface when one or more of the wearer's lower limbs are retained in the body tube and bear against the lower inner bearing surface.

20. The therapeutic body cover according to claim 10, wherein the lower inner bearing surface is provided by the lower band and, more positively when the releasable closure is sealed, against exiting of the wearer's lower limbs, by the closed structure provided by the releasable closure.
